(19) United States
(12) Patent Application Publication
(10) Pub. No.: US 2021/0068765 A1
(43) Pub. Date: Mar. 11, 2021

(54) STATE ESTIMATION APPARATUS AND NON-TRANSITORY COMPUTER READABLE MEDIUM

(71) Applicant: FUJI XEROX CO., LTD., Tokyo (JP)

(72) Inventors: Janmajay SINGH, Kanagawa (JP); Masahiro SATO, Kanagawa (JP); Takashi SONODA, Kanagawa (JP)

(73) Assignee: FUJI XEROX CO., LTD., Tokyo (JP)

(21) Appl. No.: 16/829,574

(22) Filed: Mar. 25, 2020

(30) Foreign Application Priority Data
Sep. 10, 2019 (JP) 2019-164443

(51) Int. Cl. A61B 5/00; A61B 5/0205

(52) U.S. Cl. CPC A61B 5/7275; A61B 5/0205; A61B 5/7264

(57) ABSTRACT

A state estimation apparatus includes a processor configured to estimate, from first test information obtained as a result of a first test conducted on a test target, second test information indicating a result of a second test, whether to conduct the second test being determined on a basis of a result indicated by the first test information, and estimate a state of the test target from the estimated second test information and the first test information.

### CROSS-REFERENCE TO RELATED APPLICATIONS

This application is based on and claims priority under 35 USC 119 from Japanese Patent Application No. 2019-164443 filed Sep. 10, 2019.

### BACKGROUND

### (i) Technical Field

The present disclosure relates to a state estimation apparatus and a non-transitory computer readable medium.

### (ii) Related Art

In Japanese Patent No. 4177228, a prediction apparatus that predicts a future event using an accumulated past history data is described. The prediction apparatus includes a prediction data generation unit that generates data matrices including a data matrix in which only history data is arranged as columns and a data matrix in which evaluation history data and prediction data, which is a lacking element, are arranged as columns or data matrices including a data matrix in which only history data is arranged as rows and a data matrix in which evaluation history data and prediction data, which is a lacking element, are arranged as rows. The prediction apparatus also includes a prediction unit that performs singular value decomposition on the data matrix that has been generated by the prediction data generation unit and in which only the history data is arranged as columns or rows, that estimates the lacking element, which indicates unknown prediction data, using the data matrix subjected to the singular value decomposition and the data matrix in which the evaluation history data and the prediction data are arranged as columns or rows, and that outputs the prediction data.

In Japanese Patent No. 5357871, a method for assisting a clinician in managing an acute dynamic disease of a patient using a medical apparatus including an input device that receives patient values for characterizing biological and/or physiological measured values of the patient is described. The medical apparatus further includes a calculation device that processes patient data using a model of the acute dynamic disease. The method includes supplying a plurality of first patient values to the medical apparatus and adjusting the model to dynamics of the patient using the plurality of first patient values supplied to the medical apparatus. The method also includes, in order to obtain an improved model, keeping adjusting the model to the dynamics of the patient using a latest patient value and the plurality of first patient values, the latest patient value being supplied to the medical apparatus following the plurality of first patient values, and determining an estimated patient value using the improved model. The method also includes determining an estimated value of reliability, which indicates accuracy of the estimated patient value, and determining, in order to predict the patient's recovery, a healthy area for identifying recovery in a model space including, as parameters, a concentration level of a pathogen and a response of premature promotion of inflammation included in the plurality of first patient values by supplying the plurality of first patient values to the model. The method also includes, in order to assist the clinician in managing the acute dynamic disease, outputting, to an output device of the medical apparatus, disease management information including the estimated patient value, the estimated value of reliability, and the healthy area.

In Japanese Patent No. 4449803, a time series analysis system is described. The time series analysis system includes an input device that receives measured time series data including a plurality of period components, which include a long period and a short period. The time series analysis system also includes a storage device storing time-series learning results including a short-term time-series learning result, which is a result of learning obtained by time-series learning means, and a long-term time-series learning result, which is a model optimally adjusted to the time-series data, which is a result of learning obtained by the time-series learning means, and time-series data including long-term time-series data having the long period and short-term time-series data having the short period obtained at a plurality of sets of certain time intervals. The time-series analysis system also includes the time-series learning means for learning a time-series model from the time-series data and outputting parameters of the time-series model as the time-series learning results and long-term time-series setting means for newly calculating long-term time-series data from the measured time-series data and the long-term time-series data read from the storage device, setting a model of the long-term time-series data, transmitting the model to the time-series learning means, receiving the long-term time-series learning result from the time-series learning means, and storing the long-term time-series learning result and the long-term time-series data in the storage device. The short-term time-series setting means includes a long-term time-series removal unit and a short-term time-series setting unit. The long-term time-series removal unit removes the long-term time-series data from the measured time-series data and calculates the short-term time-series data. The short-term time-series setting unit transmits the short-term time-series data to the time-series learning means, receives the short-term time-series learning result from the time-series learning means, and stores the short-term time-series learning result and the short-term time-series data in the storage device. The time-series analysis system also includes optimal model selection means for calculating predictive stochastic complexity through a stochastic process based on the long-term time-series data, the long-term time-series learning result, the short-term time-series data, and the short-term time-series learning result, selecting a learning result having time intervals with which the predictive stochastic complexity becomes smallest as an optimal model, and outputting the optimal model. The time-series analysis system also includes time-series prediction means for receiving the measured time-series data having certain time intervals and outputting time-series data a certain period of time ahead as a prediction result and an output device that outputs the prediction result.

### SUMMARY

When a first test is conducted on a test target and a second test is conducted in accordance with a result of the first test in order to detect a state of the test target, for example, it takes time and effort to conduct the second test, and accordingly it takes time to detect the state of the test target.

Aspects of non-limiting embodiments of the present disclosure relate to a state estimation apparatus and a non-transitory computer readable medium capable of accurately estimating a state of a test target without conducting a second test on the test target in accordance with a result of a first test.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided a state estimation apparatus including a processor configured to estimate, from first test information obtained as a result of a first test conducted on a test target, second test information indicating a result of a second test, whether to conduct the second test being determined on a basis of a result indicated by the first test information, and estimate a state of the test target from the estimated second test information and the first test information.

### BRIEF DESCRIPTION OF THE DRAWINGS

Exemplary embodiments of the present disclosure will be described in detail based on the following figures, wherein:

FIG. 1 is a block diagram illustrating the schematic configuration of a state estimation apparatus according to first and second exemplary embodiments;

FIG. 2 is a diagram illustrating functional blocks of the state estimation apparatus according to the first and second exemplary embodiments;

FIG. 3 is a flowchart illustrating an example of a specific process performed by the state estimation apparatus according to the first exemplary embodiment;

FIG. 4 is a diagram illustrating an example of test value information;

FIG. 5 is a diagram illustrating an example of test presence/absence information;

FIG. 6 is a diagram illustrating a method for estimating necessity for tests;

FIG. 7 is another diagram illustrating the method for estimating necessity for tests;

FIG. 8 is a diagram illustrating an example of corrected test presence/absence information;

FIG. 9 is a diagram illustrating a method for estimating whether a testee has developed sepsis;

FIG. 10 is a flowchart illustrating an example of a specific process performed by the state estimation apparatus according to the second exemplary embodiment;

FIG. 11 is a diagram illustrating a method for estimating necessity for tests;

FIG. 12 is another diagram illustrating the method for estimating necessity for tests; and

FIG. 13 is a diagram illustrating a method for estimating whether a testee has developed sepsis.

### DETAILED DESCRIPTION

### First Exemplary Embodiment

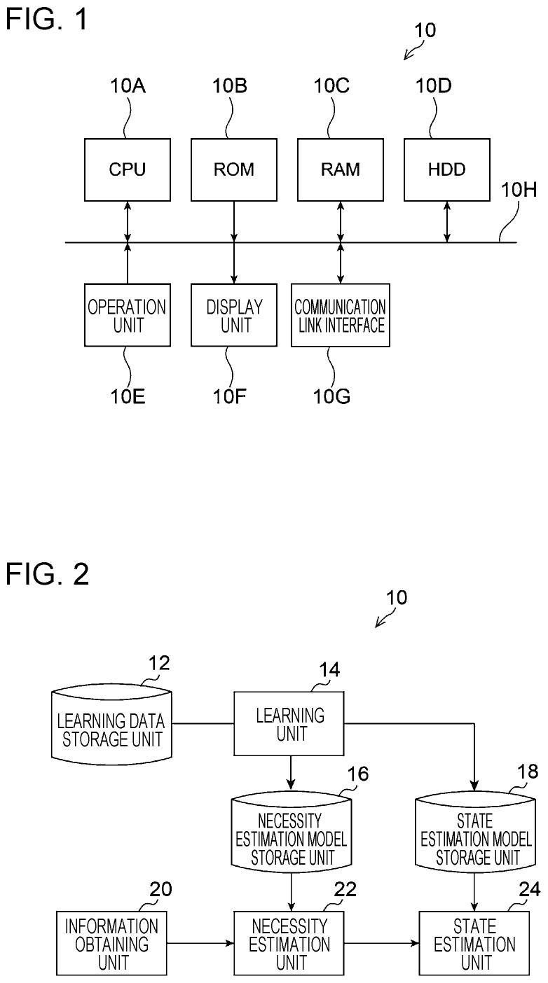

An exemplary embodiment will be described in detail hereinafter with reference to the drawings. FIG. 1 is a block diagram illustrating the schematic configuration of a state estimation apparatus according to the present exemplary embodiment.

A state estimation apparatus 10 according to the present exemplary embodiment includes a central processing unit (CPU) 10A, which is an example of a processor, a read-only memory (ROM) 10B, a random-access memory (RAM) 10C, a hard disk drive (HDD) 10D, an operation unit 10E, a display unit 10F, and a communication link interface 10G. The CPU 10A controls the entirety of the state estimation apparatus 10. The ROM 10B stores various control programs, various parameters, and the like in advance. The RAM 10C is used by the CPU 10A as a working area for executing the various programs. The HDD 10D stores various pieces of data, various application programs, and the like. The operation unit 10E includes a keyboard, a mouse, a touch panel, a stylus pen, and/or various other operation input devices and is used to input various pieces of information. The display unit 10F is a display device such as a liquid crystal display and used to display various pieces of information. The communication link interface 10G is connected to a communication link such as a network and used to communicate various pieces of data with other apparatuses connected to the communication link. The above components of the state estimation apparatus 10 are electrically connected to one another by a system bus 10H. Although the HDD 10D is used as a storage unit in the state estimation apparatus 10 according to the present exemplary embodiment, another nonvolatile storage unit such as a flash memory may be used, instead.

In this configuration of the state estimation apparatus 10 according to the present exemplary embodiment, the CPU 10A accesses the ROM 10B, the RAM 10C, and the HDD 10D, obtains various pieces of data through the operation unit 10E, and displays various pieces of information on the display unit 10F. In addition, in the state estimation apparatus 10, the CPU 10A controls communication of communication data through the communication link interface 10G.

In the state estimation apparatus 10 according to the present exemplary embodiment, the CPU 10A executes a program stored in the ROM 10B or the HDD 10D in advance to perform a process for estimating whether a testee has developed sepsis. Whether a testee has developed sepsis is an example of a state of a test target.

Next, the functional configuration of the state estimation apparatus 10 according to the present exemplary embodiment configured as described above will be described. FIG. 2 is a diagram illustrating functional blocks of the state estimation apparatus 10 according to the present exemplary embodiment. Functional units are achieved when the CPU 10A has executed a program stored in the ROM 10B or the HDD 10D in advance.

The state estimation apparatus 10 has functions of a learning data storage unit 12, a learning unit 14, a necessity estimation model storage unit 16, a state estimation model storage unit 18, an information obtaining unit 20, a necessity estimation unit 22, and a state estimation unit 24.

The learning data storage unit 12 stores a plurality of pieces of first learning data obtained from actual test data regarding testees. The plurality of pieces of first learning data include pairs of a combination of presence or absence of a heartbeat test, presence or absence of a blood pressure test, presence or absence of a blood pH test, and presence or absence of a blood glucose level test and a combination of presence or absence of necessity for a heartbeat test, presence or absence of necessity for a blood pressure test, presence or absence of necessity for a blood pH test, and presence or absence of necessity for a blood glucose level test. The learning data storage unit 12 also stores a plurality of pieces of second learning data obtained from the actual test data regarding testees. The plurality of pieces of second learning data include sets of a combination of a test value of heartbeat, a test value of blood pressure, a test value of blood pH, and a test value of blood glucose level, a combination of presence or absence of a heartbeat test, presence or absence of a blood pressure test, presence or absence of a blood pH test, and presence or absence of a blood glucose level test, and whether a testee has developed sepsis.

Heartbeat and blood pressure are an example of test items of a standard test conducted using a first test apparatus. Blood pH and blood glucose level are an example of test items of an additional test conducted using a second test apparatus. Whether to conduct the additional test is determined by a doctor on the basis of first test information.

The learning unit 14 learns a necessity estimation model in which a combination of presence or absence of necessity for a heartbeat test, presence or absence of necessity for a blood pressure test, presence or absence of necessity for a blood pH test, and presence or absence of necessity for a blood glucose level test is estimated from a combination of presence or absence of a heartbeat test, presence or absence of a blood pressure test, presence or absence of a blood pH test, and presence or absence of a blood glucose level test on the basis of the plurality of pieces of first learning data. The learning unit 14 then stores a result of the learning of the necessity estimation model in the necessity estimation model storage unit 16. As the necessity estimation model, a machine learning model such as a support-vector machine (SVM) or a deep learning model such as a deep neural network (DNN) may be used.

The learning unit 14 also learns a state estimation model in which whether a testee has developed sepsis is estimated from a combination of a test value of heartbeat, a test value of blood pressure, a test value of blood pH, and a test value of blood glucose level and a combination of presence or absence of a heartbeat test, presence or absence of a blood pressure test, presence or absence of a blood pH test, and presence or absence of a blood glucose level test on the basis of the plurality of pieces of second learning data. The learning unit 14 stores a result of the learning of the state estimation model in the state estimation model storage unit 18. As the state estimation model, a machine learning model such as a SVM or a deep learning model such as a DNN may be used.

The information obtaining unit 20 obtains test value information, which is combinations of a test value of heartbeat, a test value of blood pressure, a test value of blood pH, and a test value of blood glucose level obtained from tests conducted on a testee at different test times in the past.

The information obtaining unit 20 generates, from the obtained test value information, test presence/absence information, which is combinations of presence or absence of a heartbeat test, presence or absence of a blood pressure test, presence or absence of a blood pH test, and presence or absence of a blood glucose level test at the different test times in the past.

The necessity estimation unit 22 estimates, for each of the different test times in the past, a combination of presence or absence of necessity for a heartbeat test, presence or absence of necessity for a blood pressure test, presence or absence of necessity for a blood pH test, and presence or absence of necessity for a blood glucose level from the test presence/absence information generated by the information obtaining unit 20 for the test time using the necessity estimation model.

The state estimation unit 24 corrects the test presence/absence information at each of the different test times in the past generated by the information obtaining unit 20 using a result of estimation of a combination of presence or absence of necessity for a heartbeat test, presence or absence of necessity for a blood pressure test, presence or absence of necessity for a blood pH test, presence or absence of necessity for a blood glucose level test obtained for the test time.

The state estimation unit 24 estimates whether a testee had developed sepsis at each of the different test times in the past from the corrected test presence/absence information and the obtained test value information at the test time using the state estimation model.

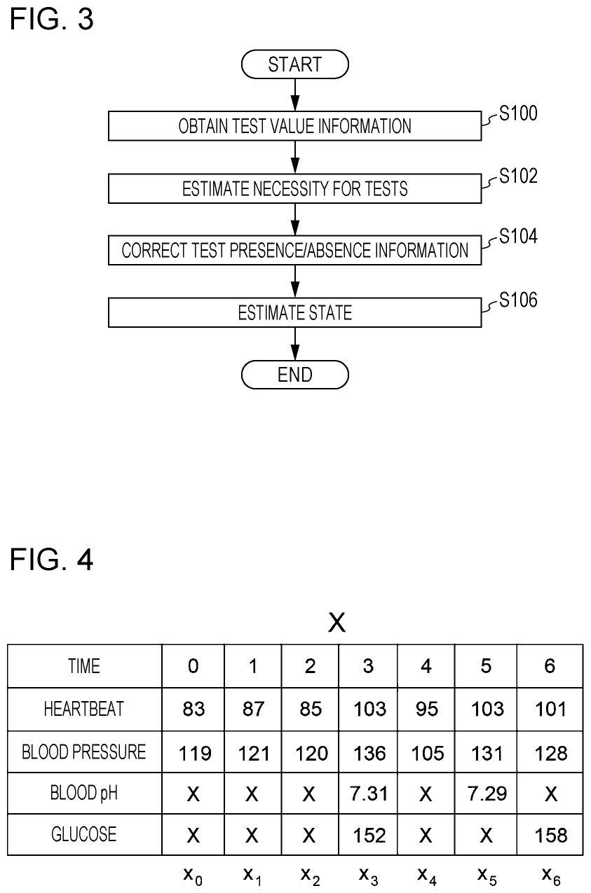

Next, a process performed by the state estimation apparatus 10 according to the present exemplary embodiment configured as described above will be described. FIG. 3 is a flowchart illustrating an example of a specific process performed by the state estimation apparatus 10 according to the present exemplary embodiment. The process illustrated in FIG. 3 starts, for example, when test value information regarding a testee at different test times in the past has been input after the learning unit 14 has learned the necessity estimation model and the state estimation model.

In step S100, the information obtaining unit 20 obtains test value information, which is combinations of a test value of heartbeat, a test value of blood pressure, a test value of blood pH, and a test value of blood glucose level obtained from tests conducted on a testee at different test times in the past.

For example, test information X illustrated in FIG. 4 is obtained. FIG. 4 is a diagram illustrating an example of test values of heartbeat, test values of blood pressure, test values of blood pH, and test values of blood glucose level of the testee at times 0 to 6. An “X” indicates that no test value was obtained, that is, no test was conducted. In addition, xₜ indicates a combination of a test value of a heartbeat, a test value of blood pressure, a test value of blood pH, and a test value of blood glucose level of the testee at a time t.

The information obtaining unit 20 generates, from the obtained test value information, test presence/absence information, which is combinations of presence or absence of a heartbeat test, presence or absence of a blood pressure test, presence or absence of a blood pH test, and presence or absence of a blood glucose level test at the different test times in the past.

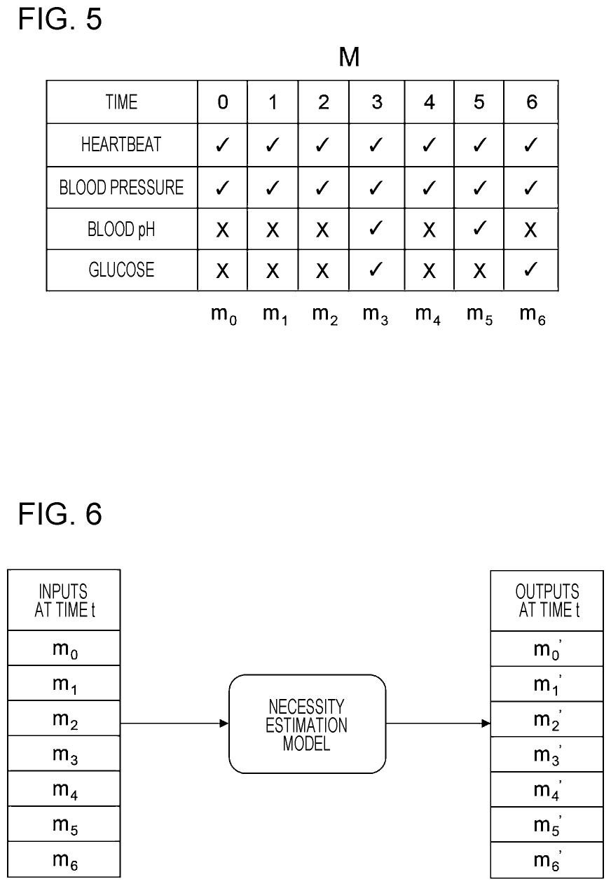

For example, test presence/absence information M illustrated in FIG. 5 is obtained. FIG. 5 is a diagram illustrating an example of presence or absence of a heartbeat test, presence or absence of a blood pressure test, presence or absence of a blood pH test, and presence or absence of a blood glucose level test of the testee at the times 0 to 6. A check indicates that a test was conducted, and an “X” indicates that a test was not conducted. In addition, mₜ indicates a combination of presence or absence of a heartbeat test, presence or absence of a blood pressure test, presence or absence of a blood pH test, and presence or absence of a blood glucose level test of the testee at the time t.

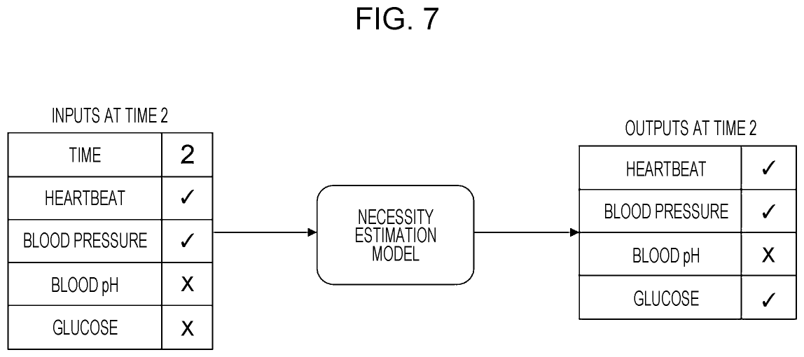

In step S102, the necessity estimation unit 22 estimates, from the test presence/absence information mₜ at each of the different test times t generated by the information obtaining unit 20, a combination mₜ′ of presence or absence of necessity for a heartbeat test, presence or absence of necessity for a blood pressure test, presence or absence of necessity for a blood pH test, and presence or absence of necessity for a blood glucose level test using the necessity estimation model (refer to FIG. 6). For example, the necessity estimation unit 22 estimates, from test presence/absence information m₂ (=[2, O, O, X, X]) at the time t=2, a combination m₂′ (=[2, O, O, X, O]) of presence or absence of necessity for a heartbeat test, presence or absence of necessity for a blood pressure test, presence or absence of necessity for a blood pH test, and presence or absence of necessity for a blood glucose level test using the necessity estimation model (refer to FIG. 7).

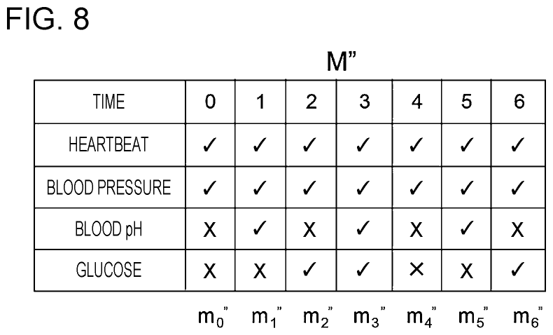

In step S104, the state estimation unit 24 corrects, using a result of the estimation of the combination mₜ′ of presence or absence of necessity for a heartbeat test, presence or absence of necessity for a blood pressure test, presence or absence of necessity for a blood pH test, and presence or absence of necessity for a blood glucose level test obtained for each of the different test times t in the past, the test presence/absence information mₜ at the test time t generated by the information obtaining unit 20. The state estimation unit 24 then generates corrected test presence/absence information M″ (refer to FIG. 8).

If a blood pH test was not conducted at the time 1 and it has been estimated that there was necessity for a blood pH test at the time 1, for example, the state estimation unit 24 corrects test presence/absence information m₁ to test presence/absence information m₁″ indicating that a blood pH test was conducted at the time 1. If a blood glucose level test was not conducted at the time 2 and it has been estimated that there was necessity for a blood glucose level test at the time 2, the state estimation unit 24 corrects the test presence/absence information m₂ to test presence/absence information m₂″ indicating that a blood glucose level test was conducted at the time 2. If a blood glucose level test was conducted at the time t and it has been estimated that there was no necessity for a blood glucose level test at the time t, however, the state estimation unit 24 does not correct the test presence/absence information mₜ.

In step S106, the state estimation unit 24 estimates whether the testee had developed sepsis for each of the different test times t from the corrected test presence/absence information mₜ″ and the obtained test value information xₜ at the test time t using the state estimation model. The state estimation unit 24 then displays results of the estimation on the display unit 10F, and the process ends.

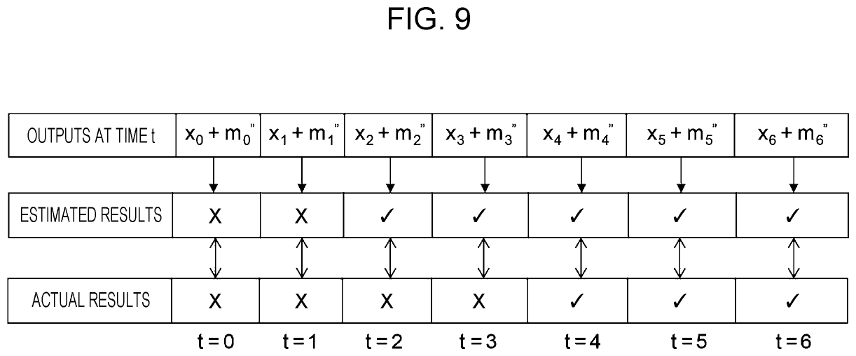

As illustrated in FIG. 9, for example, the state estimation unit 24 estimates whether the testee had developed sepsis for the time t=0 from test value information x₀ and corrected test presence/absence information m₀″ using the state estimation model. The state estimation unit 24 also estimates whether the testee had developed sepsis for the times t=1 to 6. In FIG. 9, a check indicates that the testee had developed sepsis, and an “X” indicates that the testee had not developed sepsis.

Since sepsis progresses rapidly, symptoms might appear before the additional test, which includes the blood pH test and the blood glucose level test, is conducted. In the present exemplary embodiment, whether a testee has developed sepsis can be accurately estimated using corrected test presence/absence information. As illustrated in FIG. 9, a doctor determined at the time t=4 that a testee had developed sepsis whereas the state estimation apparatus 10 estimated at the time t=2 that the testee had developed sepsis. The state estimation apparatus 10 can thus estimate at an earlier time point that a testee has developed sepsis than a doctor does on the basis of a result of an actual test.

### Second Exemplary Embodiment

Next, a second exemplary embodiment will be described. The configuration of a state estimation apparatus according to the second exemplary embodiment is the same as that of the state estimation apparatus according to the first exemplary embodiment, and description thereof is omitted while using the same reference numerals.

The learning data storage unit 12 of the state estimation apparatus 10 according to the second exemplary embodiment stores a plurality of pieces of first learning data obtained from actual test data regarding testees. The plurality of pieces of first learning data include pairs of a combination of presence or absence of a heartbeat test, presence or absence of a blood pressure test, presence or absence of a blood pH test, and presence or absence of a blood glucose level test and a combination of presence or absence of necessity for a heartbeat test, presence or absence of necessity for a blood pressure test, presence or absence of necessity for a blood pH test, and presence or absence of necessity for a blood glucose level test at a next test time. The learning data storage unit 12 also stores a plurality of pieces of second learning data obtained from actual test data regarding testees. The plurality of pieces of second learning data include sets of a combination of a test value of heartbeat, a test value of blood pressure, a test value of blood pH, and a test value of blood glucose level at a test time, a combination of presence or absence of a heartbeat test, presence or absence of a blood pressure test, presence or absence of a blood pH test, and presence or absence of a blood glucose level test at the test time, a combination of presence or absence of a heartbeat test, presence or absence of a blood pressure test, presence or absence of a blood pH test, and presence or absence of a blood glucose level test at a next test time, and whether a testee had developed sepsis at the test time.

The learning unit 14 learns a necessity estimation model in which a combination of presence or absence of a heartbeat test, presence or absence of a blood pressure test, presence or absence of a blood pH test, and presence or absence of a blood glucose level test at a next test time is estimated from a combination of presence or absence of a heartbeat test, presence or absence of a blood pressure test, presence or absence of a blood pH test, and presence or absence of a blood glucose level test at a test time on the basis of the plurality of pieces of first learning data. The learning unit 14 then stores a result of the learning of the necessity estimation model in the necessity estimation model storage unit 16. As the necessity estimation model, a machine learning model such as an SVM or a deep learning model such as a DNN may be used.

The learning unit 14 also learns a state estimation model in which whether a testee had developed sepsis at a test time is estimated from a combination of a test value of heartbeat, a test value of blood pressure, a test value of blood pH, and a test value of blood glucose level at the test time, a combination of presence or absence of a heartbeat test, presence or absence of a blood pressure test, presence or absence of a blood pH test, and presence or absence of a blood glucose level test at the test time, and a combination of presence or absence of a heartbeat test, presence or absence of a blood pressure test, presence or absence of a blood pH test, and presence or absence of a blood glucose level test at a next test time on the basis of the plurality of pieces of second learning data. The learning unit 14 then stores a result of the learning of the state estimation model in the state estimation model storage unit 18. As the state estimation model, a machine learning model such as a SVM or a deep learning model such as a DNN may be used.

The information obtaining unit 20 obtains test value information, which is combinations of a test value of heartbeat, a test value of blood pressure, a test value of blood pH, and a test value of blood glucose level obtained from tests conducted on a testee at different test times in the past.

The information obtaining unit 20 generates, from the obtained test value information, test presence/absence information, which is combinations of presence or absence of a heartbeat test, presence or absence of a blood pressure test, presence or absence of a blood pH test, and presence or absence of a blood glucose level test at the different test times in the past.

The necessity estimation unit 22 estimates, for each of the different test times in the past from the test presence/absence information generated by the information obtaining unit 20, a combination of presence or absence of necessity for a heartbeat test, presence or absence of necessity for a blood pressure test, presence or absence of necessity for a blood pH test, and presence or absence of necessity for a blood glucose level test at a next test time using the necessity estimation model.

The state estimation unit 24 determines whether the testee had developed sepsis for each of the different test times in the past from the obtained test presence/absence information and test value information at the test time and the estimated combination of presence or absence of necessity for a heartbeat test, presence or absence of necessity for a blood pressure test, presence or absence of necessity for a blood pH test, and presence or absence of necessity for a blood glucose level test at the next test time using the state estimation model.

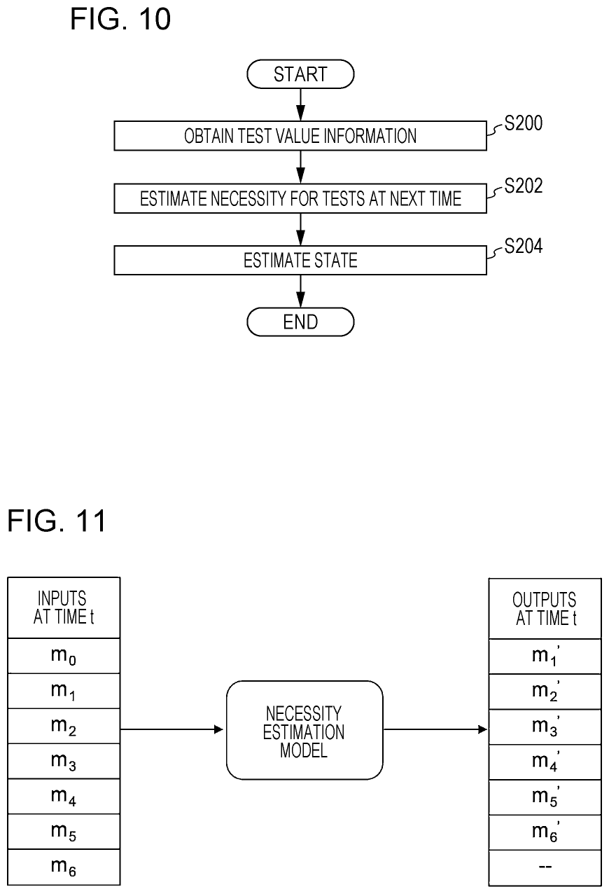

Next, a process performed by the state estimation apparatus 10 according to the second exemplary embodiment configured as described above will be described. FIG. 10 is a flowchart illustrating an example of a specific process performed by the state estimation apparatus 10 according to the second exemplary embodiment. The process illustrated in FIG. 10 starts, for example, when test value information regarding a testee at latest test times has been input after the learning unit 14 has learned the necessity estimation model and the state estimation model.

In step S200, the information obtaining unit 20 obtains test value information, which is combinations of a test value of heartbeat, a test value of blood pressure, a test value of blood pH, and a test value of blood glucose level obtained from tests conducted on a testee at the latest test times in the past. For example, the test value information X illustrated in FIG. 4 is obtained.

The information obtaining unit 20 generates, from the obtained test value information, test presence/absence information, which is combinations of presence or absence of a heartbeat test, presence or absence of a blood pressure test, presence or absence of a blood pH test, and presence or absence of a blood glucose level test at the latest test times in the past. For example, the test presence/absence information M illustrated in FIG. 5 is obtained.

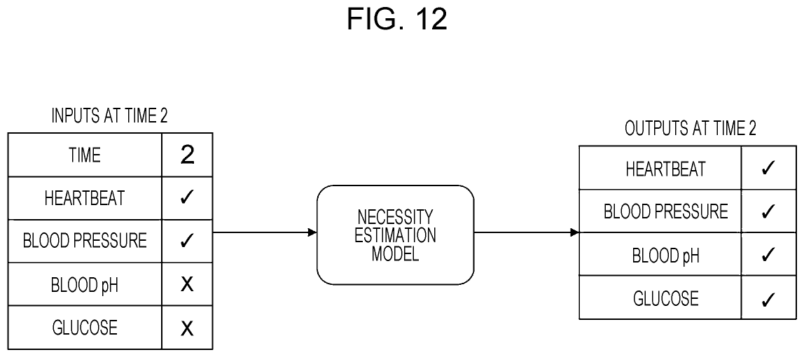

In step S202, the necessity estimation unit 22 estimates, for each of the latest test times t in the past from the test presence/absence information mₜ at the test time t generated by the information obtaining unit 20, a combination mₜ₊₁′ of presence or absence of necessity for a heartbeat test, presence or absence of necessity for a blood pressure test, presence or absence of necessity for a blood pH test, and presence or absence of necessity for a blood glucose level test at a next test time t+1 using the necessity estimation model (refer to FIG. 11). For example, the necessity estimation unit 22 estimates, from the test presence/absence information m₂ (=[2, O, O, X, X]) at the time t=2, a combination m₃′ (=[3, O, O, O, O]) of presence or absence of necessity for a heartbeat test, presence or absence of necessity for a blood pressure test, presence or absence of necessity for a blood pH test, and presence or absence of necessity for a blood glucose level test at a next time t=3 using the necessity estimation model (refer to FIG. 12).

In step S204, the state estimation unit 24 estimates, for each of the latest test times t from the obtained test presence/absence information mₜ′, the obtained test value information xₜ at the test time t, and an obtained result of the estimation of the combination mₜ′ of presence or absence of necessity for a heartbeat test, presence or absence of necessity for a blood pressure test, presence or absence of necessity for a blood pH test, and presence or absence of necessity for a blood glucose level test at the test time t, whether the testee had developed sepsis using the state estimation model. The state estimation unit 24 then displays results of the estimation on the display unit 10F, and the process ends.

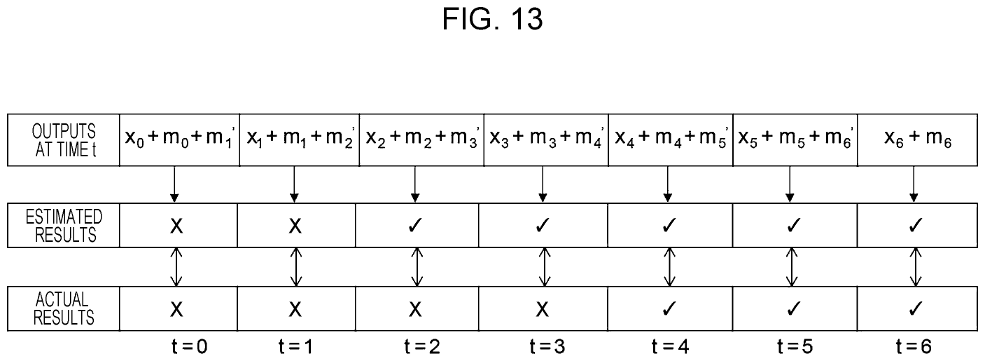

As illustrated in FIG. 13, for example, the state estimation unit 24 estimates whether the testee had developed sepsis for the time t=0 from the test value information x₀, the test presence/absence information m₀′, and the combination m₁′ of presence or absence of necessity for each test using the state estimation model. The state estimation unit 24 also estimates whether the testee had developed sepsis for the times t=1 to 6. In FIG. 13, a check indicates that the testee had developed sepsis, and an “X” indicates that the testee had not developed sepsis.

In the present exemplary embodiment, whether a testee has developed sepsis at a test time can be accurately estimated using a result of estimation of presence or absence of necessity for each test at a next test time. As illustrated in FIG. 13, a doctor determined at the time t=4 that a testee had developed sepsis whereas the state estimation apparatus 10 estimated at the time t=2 that the testee had developed sepsis. The state estimation apparatus 10 can thus estimate at an earlier time point that a testee has developed sepsis than a doctor does on the basis of a result of an actual test.

In the above exemplary embodiments, a combination of presence or absence of necessity for a heartbeat test, presence or absence of necessity for a blood pressure test, presence or absence of necessity for a blood pH test, and presence or absence of necessity for a blood glucose level test is estimated from test presence/absence information using the necessity estimation model. Only presence or absence of necessity for a blood pH test and presence or absence of necessity for a blood glucose level test included in a second test, however, may be estimated from test presence/absence information using the necessity estimation model, instead.

In addition, in the above exemplary embodiments, a heartbeat test and a blood pressure test are conducted as a first test, and a blood pH test and a blood glucose level test are conducted as the second test. A standard test other than a heartbeat test and a blood pressure test, however, may be conducted as the first test, and an additional test other than a blood pH test and a blood glucose level test may be conducted as the second test, instead. Whether to conduct the second test is determined on the basis of results of the standard test.

In addition, in the above exemplary embodiments, a combination of presence or absence of necessity for a heartbeat test, presence or absence of necessity for a blood pressure test, presence or absence of necessity for a blood pH test, and presence or absence of necessity for a blood glucose level test may be estimated from a time series of test presence/absence information using the necessity estimation model, instead.

In addition, in the above exemplary embodiments, the state estimation model may be used to estimate whether a testee has developed sepsis from a time series of test presence/absence information and a time series of test value information, instead. In this case, the state estimation model may be learned using not only test presence/absence information and test value information before a test time but also second learning data including test presence/absence information and test value information after the test time and data at the test time indicating whether the testee had developed sepsis.

In addition, in the above exemplary embodiments, the necessity estimation model may be used to estimate necessity for tests from a time series of test presence/absence information, instead. In this case, the necessity estimation model may be learned using not only test presence/absence information before a test time but also first learning data including test presence/absence information after the test time and data at the test time indicating presence or absence of necessity for each test.

In addition, although whether a testee has developed sepsis is estimated in the above exemplary embodiments, another type of state may be estimated, instead. For example, a test target may be a device outside a medical field, and whether the test target has broken down may be estimated. An effective maintenance time may then be determined from a result of the estimation of the breakdown.

In addition, in the above exemplary embodiments, the CPU 10A has been explained as an example of a processor. In the embodiments above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The process performed by the state estimation apparatus 10 according to each of the above exemplary embodiments may be a process achieved by software, hardware, or a combination of both. Alternatively, the process performed by the state estimation apparatus 10 may be stored in a storage medium as a program, and the storage medium may be distributed.

The present disclosure is not limited to the above exemplary embodiments, and the above exemplary embodiments may be modified in various ways without deviating from the scope of the present disclosure and implemented.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A state estimation apparatus comprising:
a processor configured to
estimate, from first test information obtained as a result of a first test conducted on a test target, second test information indicating a result of a second test, whether to conduct the second test being determined on a basis of a result indicated by the first test information, and
estimate a state of the test target from the estimated second test information and the first test information.

2. The state estimation apparatus according to claim 1,
wherein the processor is configured to
estimate, when estimating the second test information, necessity for each of the first and second tests at a test time from the first test information and the second test information at the test time,
correct the first and second test information at the test time using an obtained result of the estimation of the necessity for each of the first and second tests, and
estimate the state of the test target from the corrected first test information and the corrected second test information.

3. The state estimation apparatus according to claim 2,
wherein the processor is configured to estimate the state of the test target from a test value of the first test, presence or absence of the first test, and presence or absence of the second test obtained from the first and second test information.

4. The state estimation apparatus according to claim 1,
wherein the processor is configured to
estimate, when estimating the second test information, necessity for each of the first and second tests at a next test time from the first and second test information at a test time, and
estimate the state of the test target from the first and second test information at the test time and an obtained result of the estimation of the necessity for each of the first and second tests at the next test time.

5. The state estimation apparatus according to claim 4,
wherein the processor is configured to estimate the state of the test target at the test time from a test value of the first test, presence or absence of the first test, and presence or absence of the second test obtained from the first test information and the second test information at the test time and the obtained result of the estimation of the necessity for each of the first and second tests at the next test time.

6. The state estimation apparatus according to claim 2,
wherein the processor is configured to estimate the necessity for each of the first and second tests from a time series of the first test information and a time series of the second test information.

7. The state estimation apparatus according to claim 3,
wherein the processor is configured to estimate the necessity for each of the first and second tests from a time series of the first test information and a time series of the second test information.

8. The state estimation apparatus according to claim 4,
wherein the processor is configured to estimate the necessity for each of the first and second tests from a time series of the first test information and a time series of the second test information.

9. The state estimation apparatus according to claim 5,
wherein the processor is configured to estimate the necessity for each of the first and second tests from a time series of the first test information and a time series of the second test information.

10. The state estimation apparatus according to claim 2,
wherein the processor is configured to estimate the state of the test target from a time series of the first test information and a time series of the second test information.

11. The state estimation apparatus according to claim 3,
wherein the processor is configured to estimate the state of the test target from a time series of the first test information and a time series of the second test information.

12. The state estimation apparatus according to claim 4,
wherein the processor is configured to estimate the state of the test target from a time series of the first test information and a time series of the second test information.

13. The state estimation apparatus according to claim 5,
wherein the processor is configured to estimate the state of the test target from a time series of the first test information and a time series of the second test information.

14. The state estimation apparatus according to claim 6,
wherein the processor is configured to estimate the state of the test target from a time series of the first test information and a time series of the second test information.

15. The state estimation apparatus according to claim 7,
wherein the processor is configured to estimate the state of the test target from a time series of the first test information and a time series of the second test information.

16. The state estimation apparatus according to claim 8,
wherein the processor is configured to estimate the state of the test target from a time series of the first test information and a time series of the second test information.

17. The state estimation apparatus according to claim 9,
wherein the processor is configured to estimate the state of the test target from a time series of the first test information and a time series of the second test information.

18. The state estimation apparatus according to claim 1,
wherein the first test is a heartbeat test or a blood pressure test,
wherein the second test is a blood pH test or a blood glucose level test, and
wherein the state of the test target is sepsis in a testee.

19. A non-transitory computer readable medium storing a program causing a computer to execute a process for estimating a state, the process comprising:
estimating, from first test information obtained as a result of a first test conducted on a test target, second test information indicating a result of a second test, whether to conduct the second test being determined on a basis of a result indicated by the first test information; and
estimating a state of the test target from the estimated second test information and the first test information.

20. A state estimation apparatus comprising:
a means for estimating, from first test information obtained as a result of a first test conducted on a test target, second test information indicating a result of a second test, whether to conduct the second test being determined on a basis of a result indicated by the first test information; and
a means for estimating a state of the test target from the estimated second test information and the first test information.
